(19) United States
(12) Patent Application Publication
(10) Pub. No.: US 2024/0418726 A1
(43) Pub. Date: Dec. 19, 2024

(54) SPECIMEN CYTOLOGY SUPPORTING DEVICE AND METHOD ACCORDING TO CELL STAINING METHOD

(71) Applicant: THE CATHOLIC UNIVERSITY OF KOREA INDUSTRY-ACADEMIC COOPERATION FOUNDATION, Seoul (KR)

(72) Inventors: Yosep CHONG, Seoul (KR); Kwangil Yim, Seoul (KR)

(21) Appl. No.: 18/535,296

(22) Filed: Dec. 11, 2023

(30) Foreign Application Priority Data
Jun. 13, 2023 (KR) 10-2023-0075412

(51) Int. Cl. G01N 33/574; B01L 9/00; G01N 1/30; G01N 1/31; G16B 15/00; G16B 40/20

(52) U.S. Cl. CPC G01N 33/57496; B01L 9/523; G01N 1/30; G01N 1/31; G16B 15/00; G16B 40/20; G01N 2001/302

(57) ABSTRACT

A device and method for extracting a plurality of tile images from specimen cytology slide images divided according to a cell staining method, and classifying a class of at least one of a type of cancer and whether there is cancer according to a cell staining method in any specimen cytology slide image using a prediction model that has undergone annotation-based learning on the specimen cytology slide images or the tile images.

### CROSS-REFERENCE TO RELATED APPLICATION

This application claims priority from Korean Patent Application No. 10-2023-0075412, filed on Jun. 13, 2023, which is hereby incorporated by reference for all purposes as if fully set forth herein.

### BACKGROUND

### Field

Embodiments of the disclosure relate to a specimen cytology supporting device and method according to a cell staining method, supporting specimen cytology using an artificial neural network analysis technology.

### Description of Related Art

Cytology reads by staining cells using various cell staining methods and examining them under a microscope. Each cell staining method has its own advantages. Thus, a cell staining method may be selected and performed according to various situations.

However, these various cell staining methods may be a major obstacle to developing an artificial intelligence model because their colors are significantly different. Since different cell staining methods are selected according to different situations, artificial intelligence models may incorrectly learn differences according to cell staining methods.

Therefore, when developing an artificial intelligence model, it is necessary to divide various cell staining methods and develop an artificial intelligence model according to the divided cell staining methods.

### BRIEF SUMMARY

The present embodiments provide a specimen cytology supporting device and method according to a cell staining method capable of accurate specimen cytology according to a cell staining method.

The present embodiments provide a device and method for extracting a plurality of tile images from specimen cytology slide images divided according to a cell staining method, and classifying a class of at least one of a type of cancer and whether there is cancer according to a cell staining method in any specimen cytology slide image using a prediction model that has undergone annotation-based learning on the specimen cytology slide images or the tile images.

In an aspect, a specimen cytology supporting device according to a cell staining method, according to an embodiment, comprises a pre-processor extracting a plurality of tile images from a cytology slide image of a specimen divided according to the cell staining method and a classifier classifying a class of at least one of whether there is cancer and a type of cancer according to the cell staining method in any specimen cytology slide image using a prediction model that has undergone annotation-based learning using the specimen cytology slide image divided according to the cell staining method or the plurality of tile images.

In another aspect, a specimen cytology supporting method according to a cell staining method, according to another embodiment, comprises a pre-processing step extracting a plurality of tile images from a specimen cytology slide image divided according to the cell staining method and a classification step classifying a class of at least one of whether there is cancer and a type of cancer according to the cell staining method in any specimen cytology slide image using a prediction model that has undergone annotation-based learning using the specimen cytology slide image divided according to the cell staining method or the plurality of tile images.

The corresponding specimen cytology supporting device and method according to the present embodiments may provide high accuracy and efficiency, thereby significantly helping to diagnose and treat the pathology test.

The specimen cytology supporting device and method according to the present embodiments may perform accurate specimen cytology according to the type of body fluid.

### DESCRIPTION OF DRAWINGS

The above and other objects, features, and advantages of the disclosure will be more clearly understood from the following detailed description, taken in conjunction with the accompanying drawings, in which,

FIG. 1 is a block diagram illustrating a specimen cytology supporting device according to an embodiment,

FIG. 2 is a conceptual view illustrating extracting a whole slide image (WSI) image according to the present embodiment,

FIG. 3 illustrates processes of generating a plurality of tile images from an original slide image,

FIG. 4 is a schematic view illustrating a three-dimensional phase difference in a specimen cytology slide according to the present embodiment,

FIG. 5 is a diagram illustrating Z-stacking for overcoming a three-dimensional phase difference according to the present embodiment,

FIG. 6 is a diagram illustrating an example of a supervised learning method according to an embodiment,

FIG. 7A is an image illustrating labeling,

FIG. 7B illustrates images that have passed inspection and images that have not passed inspection,

FIG. 8 is a block diagram illustrating a specimen cytology supporting device according to another embodiment,

FIG. 9 illustrates images corresponding to true positive and false negative,

FIG. 10A is a flowchart illustrating an example learning algorithm for a staining method,

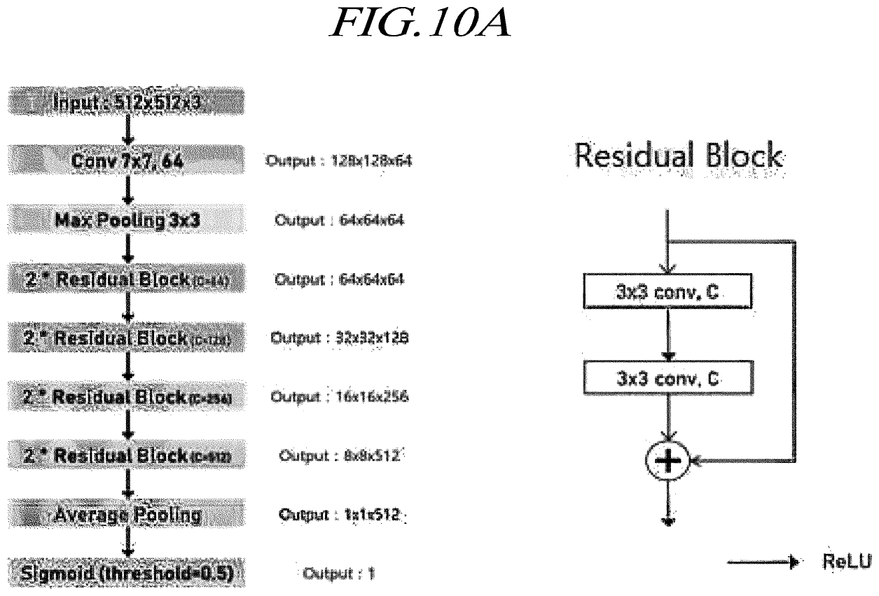

FIG. 10B is a flowchart illustrating an example learning algorithm for a type of body fluid,

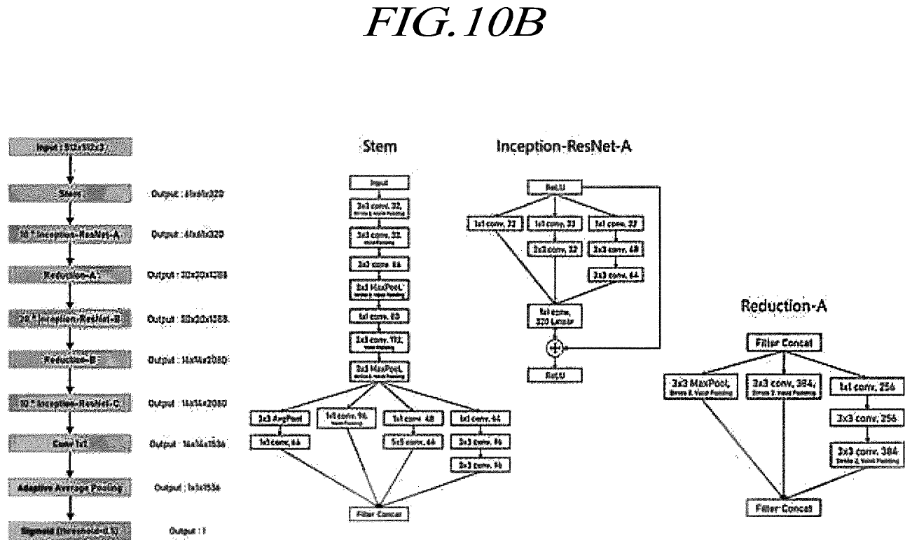

FIG. 11 is a flowchart illustrating an example specimen cytology supporting method according to another embodiment,

FIG. 12 is a block diagram illustrating a computing system according to embodiments of the disclosure, and

FIG. 13 is a block diagram illustrating a configuration of a client-server computer system according to embodiments of the disclosure.

### DETAILED DESCRIPTION

Hereinafter, embodiments of the disclosure are described in detail with reference to the accompanying drawings. In assigning reference numerals to components of each drawing, the same components may be assigned the same numerals even when they are shown on different drawings. When determined to make the subject matter of the disclosure unclear, the detailed of the known art or functions may be skipped. The terms “comprises” and/or “comprising,” “has” and/or “having,” or “includes” and/or “including” when used in this specification specify the presence of stated features, regions, integers, steps, operations, elements, and/or components, but do not preclude the presence or addition of one or more other features, regions, integers, steps, operations, elements, components, and/or groups thereof. As used herein, the singular forms “a”, “an” and “the” are intended to include the plural forms as well, unless the context clearly indicates otherwise.

Such denotations as “first,” “second,” “A,” “B,” “(a),” and “(b),” may be used in describing the components of the disclosure. These denotations are provided merely to distinguish a component from another, and the essence, order, or number of the components are not limited by the denotations.

In describing the positional relationship between components, when two or more components are described as “connected”, “coupled” or “linked”, the two or more components may be directly “connected”, “coupled” or “linked”, or another component may intervene. Here, the other component may be included in one or more of the two or more components that are “connected”, “coupled” or “linked” to each other.

When such terms as, e.g., “after”, “next to”, “after”, and “before”, are used to describe the temporal flow relationship related to components, operation methods, and fabricating methods, it may include a non-continuous relationship unless the term “immediately” or “directly” is used.

When a component is designated with a value or its corresponding information (e.g., level), the value or the corresponding information may be interpreted as including a tolerance that may arise due to various factors (e.g., process factors, internal or external impacts, or noise).

Hereinafter, embodiments are described in detail with reference to the accompanying drawings.

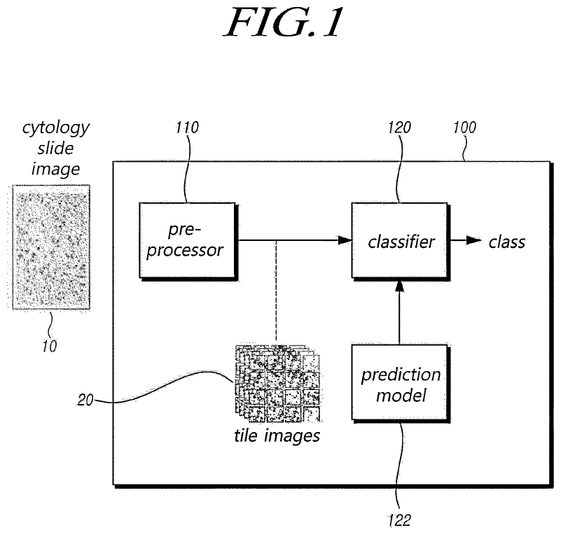

FIG. 1 is a block diagram illustrating a specimen cytology supporting device according to an embodiment.

Referring to FIG. 1, a specimen cytology supporting device 100 according to an embodiment extracts a plurality of tile images 20 from a specimen cytology slide image 10 divided according to a cell staining method, and classifies a class of at least one of whether there is cancer or a type of cancer according to a cell staining method from the specimen cytology slide image 10 divided according to any cell staining method using a prediction model 122 in which annotation-based learning is performed on the specimen cytology slide image 10 divided according to the cell staining method or the tile images 20.

The specimen cytology supporting device 100 according to an embodiment is a device for supporting collecting cytology specimens or glass slide specimens by the cell staining method, body fluid type, normal and cancer diagnosis class, and carcinoma, and dividing normal/cancer or detailed carcinoma according to the cell staining method.

The specimen cytology supporting device 100 according to an embodiment may construct a suitable learning dataset and evaluation dataset as described below to develop a prediction model 122 that has undergone annotation-based learning, which is, e.g., an artificial intelligence analysis model, train and evaluate the prediction model 122 using the learning dataset and the evaluation dataset, and classify a class of at least one of whether there is cancer and the type of cancer according to the cell staining method from the specimen cytology slide image 10 divided according to any cell staining method, using the trained prediction model 122.

The specimen cytology supporting device 100 according to an embodiment includes a pre-processor 110 for pre-processing the specimen cytology slide image 10 divided according to the cell staining method, and a classifier 120 for classifying one class from the specimen cytology slide image 10 divided according to any cell staining method using the prediction model 122 that has undergone annotation-based learning.

The type of body fluid may be at least one of respiratory specimens, pleural fluid, ascites, urine, and fine needle aspiration cytology, but is not limited thereto.

For example, if the specimen is a respiratory specimen, the type of cancer may be lung cancer.

Currently, lung cancer tumors are confirmed by cytology obtained by sputum, bronchoscopy, and bronchial alveoli or histopathology obtained by bronchoscopy biopsy. Cytology is less invasive than histopathology. However, since the cytology cannot observe the structure of the cell, the difficulty of diagnosis is high. In particular, various types of tumors occur in the lungs, and cytology has difficulty in developing detailed types.

When the specimen is pleural fluid, the type of cancer may be lung cancer and breast cancer.

Currently, pleural fluid tests usually use cytology, which performs diagnosis by smearing pleural fluid on a slide and then examining it with a microscope, as a basic screening test. However, when reactive mesothelial cells are activated in an environment where pleural membranes are stimulated, such as inflammation, it becomes difficult to distinguish them from malignant cells, so the sensitivity of cytology is reported to be very low. These false negative test results cause the patient's loss of treatment opportunities, bad prognosis, and increase medical expenses due to missing the treatment timing or failure to properly diagnose recurrence.

Currently, other screening tests and tumor markers using molecular pathology techniques that may replace cytology are being developed, but they are not widely used as tests to replace cytology due to their high cost and low sensitivity and accuracy than expected.

Recently, artificial neural network image analysis technology has been greatly developed and has been applied to classification, detection, and measurement areas using various digital pathological images, showing quite good results. If this is applied to pleural fluid cytology specimen analysis, it may be expected that a more accurate early diagnosis will be possible with a very low cost and simple non-invasive test.

If this is applied to pleural fluid cytology, it is expected that more accurate early diagnosis will be possible with simple non-invasive tests at a very low cost.

When there are multiple specimens, the types of cancer may be ovarian cancer, colon cancer, gastric cancer, and pancreatic cancer, and if it is ovarian cancer, the types of cancer may be serous cancer, mucous cancer, endometrial cancer, and transparent cell cancer.

Ovarian cancer has the poorest prognosis among gynecological cancers and the lowest survival rate (5-year survival rate of 15-55%) among gynecological cancers, and is relatively increasing. However, specimen cell diagnosis is essential in ovarian cancer, but there are no effective screening tests or markers. Further, in ovarian cancer, peritoneal metastasis occurs faster than other cancers and may be essential for staging. Various molecular pathology markers continue to be developed but, because there are no specific cancer markers, they are expensive and show performance similar to specimen cytology.

Cytology is simple and inexpensive minimally invasive, but suffers from low sensitivity. Further, when immunohistochemical staining is required, such as reactive mesothelial cells, endometriosis, endosalpingiosis, and the like are difficult to screen.

Further, colon cancer, gastric cancer, and pancreatic cancer, along with ovarian cancer, are carcinomas that are subject to easy intraperitoneal metastasis, SO the metastasis is evaluated with a specimen.

Currently, specimen cytology usually uses specimen cytology, which performs diagnosis by smearing specimen fluid on a slide and then examining it under a microscope, as a basic screening test.

However, when reactive mesothelial cells are activated in an environment where peritoneum is stimulated, such as inflammation, it becomes difficult to distinguish them from malignant cells, so the sensitivity of specimen cytology is reported to be very low. These false negative test results cause the patient's loss of treatment opportunities, bad prognosis, and increase medical expenses due to missing the treatment timing or failure to properly diagnose recurrence.

When the specimen is urine, the type of cancer may be bladder cancer.

Cytology using urination and bladder washing is the primary screening test for bladder cancer and is a very difficult test for pathologists due to its low accuracy. Recently, researchers have begun to apply artificial intelligence technology to analyze cytological specimens.

When the specimen is fine needle aspiration cytology, the type of cancer may be thyroid gland, salivary gland, lung cancer, pancreatic cancer, and lymphoma.

Currently, the thyroid cancer test uses cytology, which performs diagnosis by stabbing cells with a fine needle, smearing them on a slide, and examining them under a microscope, as a basic screening test.

Currently, the salivary gland, lung cancer, pancreatic cancer tumors, and lymphomas are confirmed by fine needle aspiration cytology or core needle biopsy. The fine needle aspiration cytology is less invasive than the core needle biopsy. However, since the fine needle aspiration cytology cannot observe the structure of the cell, the difficulty of diagnosis is high. In particular, various types of tumors occur in the salivary glands, and cytology has difficulty in diagnosing detailed types.

Currently, other screening tests and tumor markers using molecular pathology techniques that may replace specimen cytology are being developed, but they are not widely used as tests to replace specimen cytology due to their high cost and low sensitivity and accuracy than expected.

Recently, artificial neural network image analysis technology has been greatly developed and has been applied to classification, detection, and measurement areas using various digital pathological images, showing quite good results. If this is applied to specimen cytology, it is expected that more accurate early diagnosis will be possible with simple non-invasive tests at a very low cost.

The pre-processor 110 extracts a plurality of tile images 20 from the specimen cytology slide image 10 divided according to the cell staining method.

The pre-processor 110 extracts the tile images 20 from the specimen cytology slide image 10 divided according to the cell staining method using an image processing technique such as an image segmentation and fusion technique, and adjusts the size and resolution of the tile image to enable efficient learning and prediction.

The classifier 120 classifies a class of at least one of whether there is cancer or the type of cancer in the specimen cytology slide image 10 divided according to any cell staining method using the prediction model 122 that has undergone annotation-based learning using the specimen cytology slide image 10 divided according to the cell staining method or two or more tile images 20.

In this case, the specimen cytology slide image 10 divided according to the cell staining method may be, e.g., a slide image of body fluids of the specimen, and may be a whole slide image (WSI).

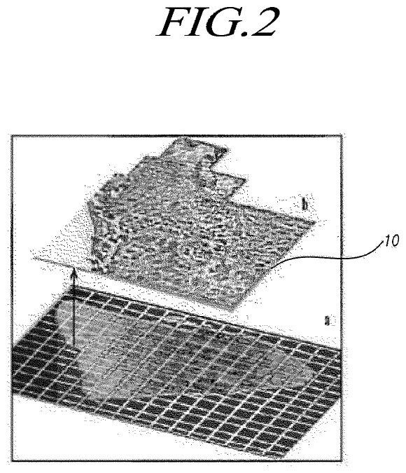

FIG. 2 is a conceptual view illustrating extracting a whole slide image WSI.

As illustrated in FIG. 2, the whole slide image is extracted by smearing and capturing or scanning the glass slide of the specimen. The extracted whole slide image may be an unprocessed original slide image.

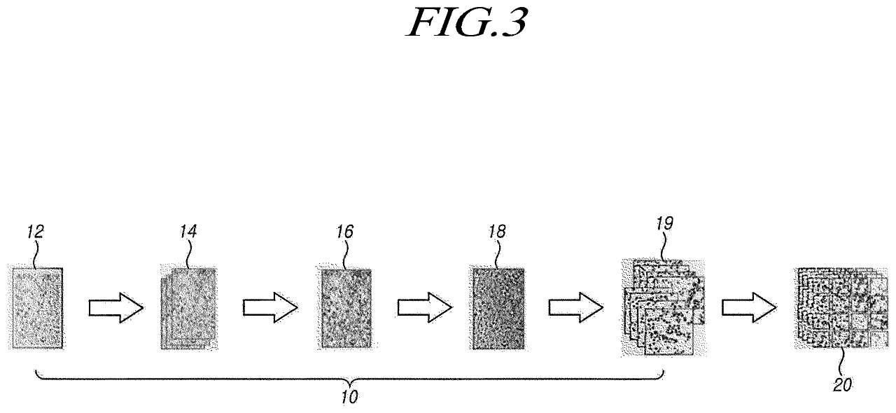

FIG. 3 illustrates processes of generating a plurality of tile images from an original slide image. The processes of FIG. 3 include a Z-stacking or focus stacking process and a color normalization process.

Referring to FIG. 3, the specimen cytology slide image 10 divided according to the cell staining method is obtained from the original slide image 12 obtained by spearing and capturing or scanning on the glass slide of the specimen using the Z-stacking or focus stacking technique.

Specifically, the specimen cytology slide image 10 divided according to the cell staining method may be obtained by synthesizing the images 14 focused at different phases from the original slide image 12 into one image 16 through secondary post-processing using Z-stacking or focus stacking technique.

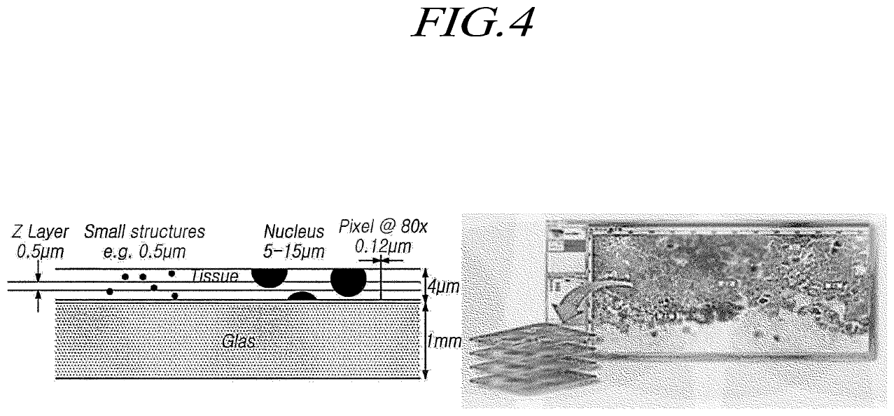

For example, the cytology whole slide image WSI has a three-dimensional structure in the slide due to the characteristics of the cell specimen as illustrated in FIG. 4. Therefore, it may be necessary to scan the cytology whole slide image WSI including two or more images 14 focused at different phases at a high magnification, e.g., a high magnification of 40×, to observe cell nuclei, nucleoplasm, cytoplasm, etc.

As a specific example, in order to overcome the three-dimensional phase difference, two or more, e.g., five to 20 images focused on different phases may be obtained, stored, and displayed, or may be synthesized into one image 16 through secondary post-processing.

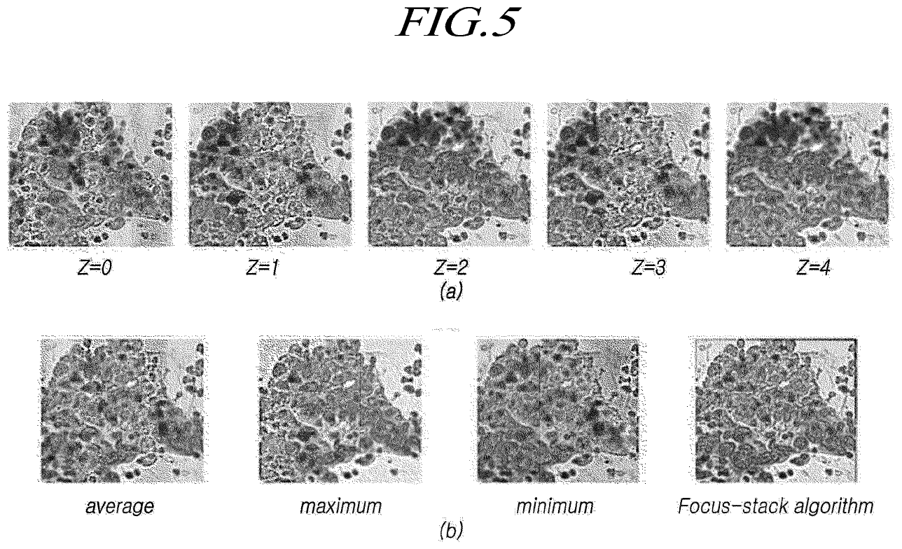

As illustrated in FIG. 5, e.g., five images focused at different phases (z=0 to z=4) may be obtained and all used, or for example, the images focused at different phases (z=0 to z=4) may be synthesized into one image 16 through secondary post-processing such as averaging, maximizing, minimizing, or applying a focus-stacking algorithm, thereby obtaining the specimen cytology slide image 10 divided according to the cell staining method.

Next, as illustrated in FIG. 3, through research on standardization technology of scanned digital images, an image 18 may be obtained by color normalization that makes the colors of stains that may look different due to various staining conditions similar to each other. Further, in the specimen cytology slide image 10 divided according to the cell staining method, various artifacts that may occur during the slide preparing process, e.g., tissue dropout, crushing, air bubble, dust, foreign substances, and the like, may be corrected using an image processing technique.

The specimen cytology slide image 10 divided according to the cell staining method may be any one of the original slide image 12 and images 14 focused at different phases from the original slide image 12, the one image 16 synthesized through secondary post-processing, and the color normalized image 18.

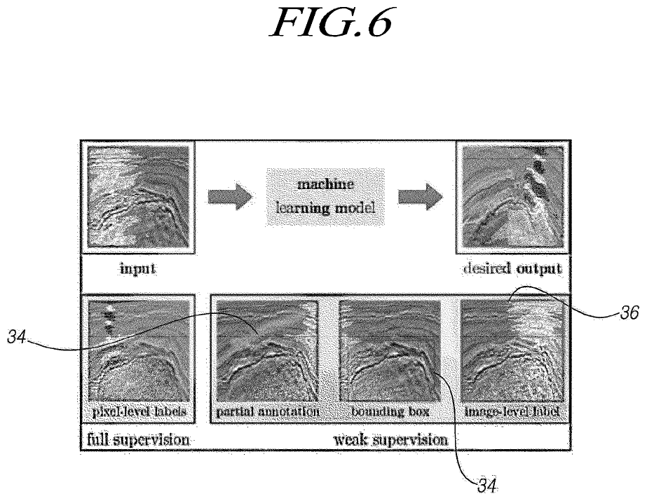

Further, the specimen cytology slide image 10 divided according to the cell staining method may be a slide image obtained without some of the processes described with reference to FIG. 6. For example, the specimen cytology slide image 10 divided according to the cell staining method may be one image 16 synthesized through secondary post-processing without color normalization. The specimen cytology slide image 10 divided according to the cell staining method may be an image 18 obtained by color-normalizing the original slide image 12 without applying the images 14 focused at different phases from the original slide image 12 and one image 16 synthesized through secondary post-processing.

The extracted lesion area, e.g., the cancer area, may be cut to a specific size to be extracted as a plurality of tile images or structured patch data that may be learned. Further, the class annotation information marked to the specimen cytology slide image divided according to the cell staining method may be assigned to all tile images or patch data extracted from the specimen cytology slide image divided according to the cell staining method.

Meanwhile, the plurality of tile images may be images having a size smaller than the specimen cytology slide image divided according to the cell staining method.

For this reason, the specimen cytology slide image 10 divided according to the above-described cell staining method or the cytology image including the tile images 20 may be stored as a file having a capacity of 5 to 10 times that of a general histopathological image. For example, the histopathological image may have an average of 10 Gb compared to the average of 1 Gb.

The pre-processor 110 may generate a plurality of tile images 20 based on a sliding window algorithm. In other words, the pre-processor 110 may generate a plurality of tile images by extracting a portion overlapping the sliding window as a tile image on the specimen cytology slide image divided according to the cell staining method, then moving the position of the sliding window, and then repeating the extraction of the tile image.

For example, the plurality of tile images may be RGB images having a red (R) channel, a blue (B) channel, and a green (G) channel.

When the classifier 120 uses the prediction model 122 in which annotation-based learning is performed using the specimen cytology slide image 10 divided according to the cell staining method or two or more tile images 20, annotation-based learning allows an expert to directly annotate the extracted lesion area, e.g., a cancer area, so that the prediction model 122 is accurately trained.

The prediction that has undergone model 122 annotation-based learning may perform learning by adding one or more of partial annotation 32 indicating the cancer area in a line form, bounding box annotation 34 indicating the cancer area in a box form, and image-level label 36 indicating the whole image to the specimen cytology slide image 10 divided according to the cell staining method or the plurality of tile images 20 used for learning. The shape of the annotation is not limited to a line shape or a bounding box, and may vary. For example, the shape of the annotation may vary, such as a line shape, an elliptical shape, and a parentheses shape.

In other words, the prediction model 122 that has undergone the annotation-based learning may perform the learning through the specimen cytology slide image 10 divided according to the cell staining method or the plurality of tile images 20 to which the annotation indicating the cancer area is added.

This prediction model 122 may be a cytology slide-based neoplasm prediction model. For example, the prediction model may be developed as a weakly-supervised learning model capable of predicting a result in a square tile unit using a slide unit label using a square tile detection algorithm in which tissue is present in the specimen cytology slide image WSI divided according to the whole cell staining method and the slide unit label for neoplasm.

Specifically, a loss function which is known to work well in classification model learning may be applied to model learning. Further, model learning may be performed based on an annotation on whether there is cancer on a slide-by-slide basis. The annotation may be the partial annotation 32, the bounding box annotation 34, and the image level label 36 as described above.

There are various cell staining methods used in cytology. Further, various cell staining methods have been developed and used to analyze specific diseases or cell structures. This may vary depending on the type of cell staining method and the purpose of the test.

For example, the cell staining method may be one of Giemsa staining, hematoxylin-eosin (H&E) staining, Papanicolaou (PAP) staining, and Diff-quik staining.

Giemsa staining is used to stain cell nuclei and extracellular structures. Giemsa staining is widely used to diagnose blood-related diseases and helps visualize cell structures.

H&E staining uses hematoxylin, which stains the cell nucleus blue, and eosin, which stains the outside of the cell red to differentiate the detailed structure of the cell. H&E staining is generally used in biopsy and helps determine the structure and condition of cells and tissues.

PAP staining is used to visualize cell structure. PAP staining is used to screen the shape and structure of cells in cell specimens to diagnose sexual dysfunction and cancer.

Diff-quik staining is one of the cell staining methods and is a technique for staining cell specimens in a quick and simple way. Diff-quik staining is mainly used in nuclear cytology and helps evaluate cell abnormalities by visualizing the cell nucleus structure and the outside of the cell.

Diff-quik staining is based on the Giemsa staining, and may complete cell staining in a simpler and faster process than general Giemsa staining. Diff-quik staining includes lightly washing the cell specimen with water, immersing the cell slide in the Diff-quik staining solution for several seconds, and then locally washing and staining.

A plurality of prediction models 122 that have undergone annotation-based learning may be present for each cell staining method of the specimen and for each type of cancer. In this case, the classifier 120 may classify a class of at least one of whether there is cancer or the type of cancer according to each cell staining method in any specimen cytology slide image using the prediction model 122 that has undergone the annotation-based learning for each cell staining method of the specimen and each type of cancer.

The prediction model 122 may perform a data gathering step and, as described above, the data pre-processing step, the model training step for performing annotation-based learning or weakly-supervised learning, and a model validation step for validating the trained model, analyze the specimen cytology slide image divided according to any cell staining method when the specimen cytology slide image divided according to any cell staining method is input, and classify a class of at least one of whether there is cancer and the type of cancer according to the cell staining method. The prediction model 122 may also identify the position arm using of the an image classification algorithm.

The prediction model 122 may divide whether there is cancer or the type of cancer according to the cell staining method into a respective class and define, and when receiving a request for the classification of the class for the specimen cytology slide image 10 divided according to any cell staining method, may classify the class as a result of annotation-based learning.

Further, the prediction model 122 may be generated using an ensemble learning method. One prediction model 122 may classify whether there is cancer and the type of cancer according to the above-described cell staining. There may be prediction models 122 of specimens trained for each cancer, and each prediction model 122 of specimens trained for each cancer may determine whether the cancer corresponds to the corresponding cancer, and the results of prediction by the prediction models may be compiled to classify whether there is cancer and the type of cancer according to the cell staining method.

For example, for the specimen cytology slide image 10 divided according to any cell staining method, the prediction model 122 trained with a specific cancer may classify the cancer as the corresponding cancer, and the prediction model 122 trained to classify detailed cancers may classify the carcinomas as one of the detailed cancers. The prediction model 122 trained with other cancers may classify the cancer as not corresponding to the corresponding cancer.

Further, the prediction model 122 may divide whether there is cancer or the type of cancer according to the type of body fluid into a respective class and define, and when receiving a request for the classification of the class for the specimen cytology slide image 10 divided according to any body fluid type, may classify the class as a result of annotation-based learning.

In other words, there are a plurality of prediction models that have undergone annotation-based learning according to the body fluid types of the specimen and the cancer types.

The classifier 120 may classify a class of at least one of whether there is cancer or the type of cancer according to the body fluid type in any specimen cytology slide image using the prediction model that has undergone the annotation-based learning for the body fluid type of the specimen and each type of cancer.

In this case, the specimen according to the type of body fluid may be at least one of a respiratory specimen, pleural fluid, ascites, urine, and fine needle aspiration cytology.

Whether there is cancer according to the type of body fluid may be divided into positive or negative.

If the specimen is a respiratory specimen, the type of cancer may be lung cancer, if the specimen is pleural fluid, the type of cancer may be lung cancer and breast cancer, if there are a plurality of specimens, the type of cancer may be ovarian cancer, colon cancer, gastric cancer, and pancreatic cancer, if the cancer is ovarian cancer, the cancer may be serous cancer, mucous cancer, endometrial cancer, or transparent cell cancer, if the specimen is urine, the type of cancer may be bladder cancer, and if the specimen is fine needle aspiration cytology, the type of cancer may be thyroid gland, salivary gland, lung cancer, pancreatic cancer, lymphoma, but is not limited thereto.

This prediction model 122 may classify only whether there is cancer according to the type of body fluid, or may classify the type of cancer as well as whether there is cancer.

For example, the prediction model 122 may classify only whether there is cancer according to the type of body fluid in the specimen, or may classify the type of cancer as well as whether there is cancer according to the type of body fluid.

For example, when the specimen is a respiratory specimen, the prediction model 122 may include a lung cancer classification model according to the cancer.

When the specimen is pleural fluid, the prediction model 122 may include a lung cancer and breast cancer classification model according to the cancer.

When there are a plurality of specimens, the prediction model 122 may include a classification model for ovarian cancer, colon cancer, gastric cancer, and pancreatic cancer according to the cancer. In particular, in the case of ovarian cancer, the prediction model 122 may include a serous cancer, mucous cancer, endometrial cancer, or transparent cell cancer classification model.

When the specimen is urine, the prediction model 122 may include a bladder cancer classification model according to the cancer.

When the specimen is fine needle aspiration cytology, the prediction model 122 may include of a thyroid gland, salivary gland, lung cancer, pancreatic cancer, and lymphoma classification model according to the cancer.

The prediction model 122 may be a model for dividing each cancer. For example, when there are a plurality of specimens, the prediction model 122 may be a model for distinguishing whether it is ovarian cancer or not. The prediction model 122 may be a model for distinguishing whether it is colon cancer or not. The prediction model 122 may be a model for distinguishing whether it is gastric cancer or not. The prediction model 122 may be a model for distinguishing whether it is pancreatic cancer or not. In the case of ovarian cancer, another prediction model 122 may distinguish serous cancer, mucous cancer, endometrial cancer, or transparent cell cancer.

Even when the specimens are different, each prediction model 122 may be a model for distinguishing various types of cancers.

As described above, the prediction model 122 performs the data gathering step and the data pre-processing step, the model training step for performing annotation-based learning or weakly-supervised learning, and the model validation step of validating the trained model. Hereinafter, these processes and the results will be exemplarily described.

### Selection of Raw Data

Body fluid cytology specimens are non-gynecological cytology and are largely composed of five specimens, respiratory specimens, pleural fluid, ascites, urine, and fine needle aspiration cytology (FNAC) tests, and learning data were constructed as illustrated in Table 1 using the same number of specimens to suit the development of artificial intelligence models suitable for each (a total of 5,500 cases, 11,000 cases for each body fluid).

TABLE 1

| Sample type | Diagnosis | WSIs | Image patches |
|---|---|---|---|
| Respiratory tract | Lung ca | 716 | 10069 |
| | Benign | 557 | 20521 |
| Pleural fluid | Lung ca. Breast ca. | 501 | 29952 |
| | Benign | 567 | 19909 |
| Ascites | Ovary ca. Stomach ca. | 524 | 24542 |
| | Colon ca. Pancreas ca. | | |
| | Benign | 507 | 21527 |
| Urine | Bladder ca. | 503 | 20105 |
| | Benign | 503 | 20382 |
| FNA | Thyroid ca. Salivary gland ca. | 539 | 19925 |
| | Lung ca. Pancreas ca. | | |
| | Lymphoma/meta | | |
| | Benign | 589 | 20105 |
| Total | | 5506 | 207037 |

In the case of most cytology, labeling was practically difficult in units of individual cells or cell clusters, and since most of the artificial intelligence models currently developed were in the form of classification models in units of image patches, they were extracted and constructed in the form of image patches suitable for model development.

Carcinomas relatively commonly observed for each type of body fluid were included. The specimens for degree management gathered by diagnostic review conducted anonymously and by specialists through the Korean Society for Cytopathology's degree management program in 210 pathology examination rooms and eight flagship local hospitals were 20%, and the remaining 80% of the data were specimens from three organizations including the Catholic University of Korea's Uijeongbu St. Mary's Hospital, Yonsei University's Severance Hospital, and the National Cancer Center.

In the focus stacking method optimized for the quality of slide staining of each organization and the smearing state of the individual specimen, a minimum of 3 layers to a maximum of 6 layers were selected to minimize errors in shape acquisition such as out-of-focus and over-overlapping of images.

### Collecting, Refining, Annotation/Labeling Procedure

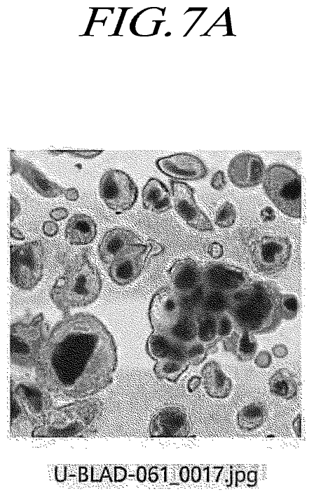

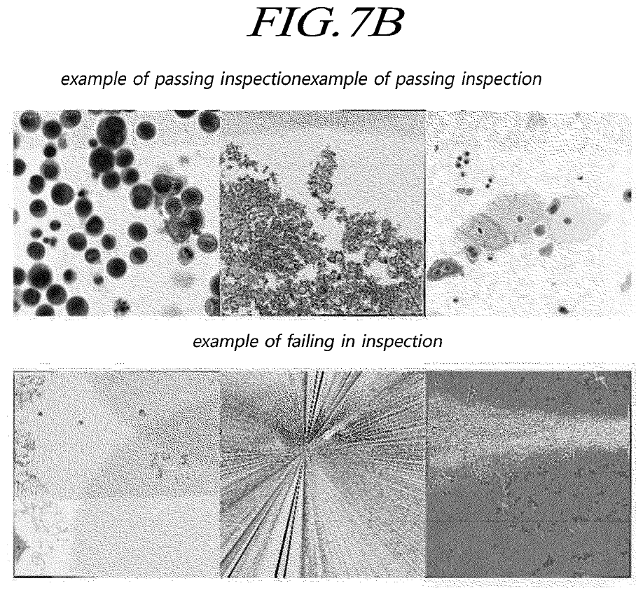

Body fluid cytology specimens or glass slide specimens were collected by the cell staining method, body fluid type, normal and cancer diagnosis class, and carcinoma, and cytology diagnosis and histopathology diagnosis were reviewed through re-examination, and scanned with a digital slide scanner. Thereafter, the digital image (svs or mrxs file), which is scanned raw data, was qualitatively reviewed, and was then subjected to Z-stacking image synthesis (extended Z-stacking image generation), removal of foreign matter and unfocused areas, and color normalization, as the data refining process, and an image patch having a size of 1024×1024 pixels was extracted. Thereafter, a standardized jpeg format was obtained through a resizing process of reducing the size to 256×256 pixels after quality inspection. The data standardized in the jpeg format was labeled for training the artificial intelligence model. FIG. 7A is an image illustrating labeling.

### Collecting, Refining, Annotation/Labeling Standard

Labeling was first performed by each organization in the slide unit (WSI) according to the diagnostic class classification standard at the time of scanning, and in the case of image patches extracted from the whole slide image (WSI), normal slides all were assigned normal class annotation in the image patches, and the cancer was reclassified as normal or cancer class after review on the extracted image patches by two or more experts (cell pathologists and cell pathology specialists), during which course data not meeting the quality standard was excluded. Labeling was basically based on the histopathological diagnosis of the same patient corresponding to the cell slide, but the data was used as source data if there was no disagreement between experts in the clinical situation or cytology findings even without histopathological diagnosis.

When there were different opinions between experts in the expert review of two of the image patches extracted from the whole slide image of the cancer class, they were excluded from the learning image dataset.

The labeling data was configured as illustrated in Table 2.

TABLE 2

| Item | Type | Description | Range |
|---|---|---|---|
| Dataset | Number | Staining | 01: H&E |
| strain | | method | 02: PAP |
| | | | 03: DipQuik |

In other words, the labeling data includes H&E staining, PAP staining, and Diff-quik staining as cell staining methods.

### Inspection

Before submitting the data, all of the three organizations conducted their own quantitative inspection and semantic suitability evaluation on the per-quality characteristic items of the data. For the constructed data, all image patches were manually cross-validated by the Catholic University of Korea Uijeongbu St. Mary's Hospital, the National Cancer Center, and Asan Medical Center in Seoul. FIG. 7B illustrates images that have passed inspection and images that have not passed inspection.

TABLE 3

| quality | | measurement | quantitative |
|---|---|---|---|
| characteristic | item | index | target |
| diversity | class | ratio | 01: normal 45-55% |
| | | | 02: malignant 45- |
| | | | 55% |
| | body fluid type | minimum | 01: respiratory |
| | | amount | specimen 1000 |
| | | | count (20000 |
| | | | million sheets), |
| | | | 02: pleural fluid |
| | | | 1000 count (20000 |
| | | | million sheets), |
| | | | 03: ascites 1000 |
| | | | count (20000 |
| | | | million sheets), |
| | | | 04: fine needle |
| | | | aspiration |
| | | | cytology 1000 |
| | | | count (20000 |
| | | | million sheets), |
| | | | 05: urine 1000 |
| | | | count (20000 |
| | | | million sheets) |
| | diagnosis name | type | includes at least |
| | | | eight carcinomas |
| | | | among 01: lung |
| | | | cancer, 02: breast |
| | | | cancer, 03: |
| | | | ovarian cancer, |
| | | | 04: stomach |
| | | | cancer, 05: colon |
| | | | cancer, 06: |
| | | | pancreatic cancer, |
| | | | 07: bladder |
| | | | cancer, 08: |
| | | | thyroid cancer, |
| | | | 09: |
| | | | lymphoma/metastatic |
| | | | cancer, and 10: |
| | | | salivary gland |
| | | | cancer |
| | | minimum | at least 150 |
| | | amount | counts for each |
| | | | type of diagnosis |
| | smear/liquid | ratio | 01: liquid |
| | cell | | 02: spear |
| | scanner type | type/ratio | includes at least |
| | | | three types among |
| | | | 01: 3DHistech, 02: |
| | | | Leica AT2, 03: |
| | | | Hammamatsu, 04: |
| | | | Roche, and 05: |
| | | | Philips(at least |
| | | | 1% or more) |
| | staining method | type/ratio | includes two or |
| | | | more staining |
| | | | methods among H&E, |
| | | | PAP, Diff-quik |
| | | | (at least 5% or |
| | | | more) |
| | primary/ | ratio | 01: primary 30-70% |
| | metastatic | | 02: metastatic 30- |
| | | | 70% |
| division | division | Accuracy (%) | 99% |
| accuracy | accuracy | | |
| meaning | classification | Accuracy (%) | 99% |
| accuracy | label accuracy | | |
| validity | pleural fluid- | Accuracy | 0.8 or more |
| | lung cancer and | | |
| | breast cancer | | |
| | cell diagnosis | | |
| | classification | | |
| | model | | |
| | urine-bladder | Accuracy | 0.8 or more |
| | cancer cell | | |
| | diagnosis | | |
| | classification | | |
| | model | | |

### Learning Model

In order to classify the cytology image as normal/abnormal, an image classification function is required to display and classify the probability of the class.

The EfficientNet algorithm extracted features of the image, predicted the possibility of presence of normal and abnormal using the extracted features, and output a class classification.

A learning model was developed by dividing data into training, validation, and evaluation in the form illustrated in Table 4 below.

TABLE 4

| | Train | Valid | Test | Total |
|---|---|---|---|---|
| HE | 19385 | 2424 | 2427 | 24236 |
| (cell block) | | | | |
| PAP | 19389 | 2422 | 2429 | 24240 |
| Total | 38774 | 4846 | 4856 | 48476 |

In order to validate the learning model, the whole data was divided into learning (80%), validation (10%), and test (10%), and learning and testing were performed.

An accuracy was calculated according to the result values divided through the artificial intelligence prediction model 122.

For example, the prediction model 122 may define an optimization parameter for each model for enhancing accuracy. Further, the prediction model 122 may define parameters for comparing algorithms suitable for medical data characteristics and optimizing performance.

For example, the main optimization parameters may be shown as in Table 5.

TABLE 5

| main optimization parameters | description |
|---|---|
| EPOCH_COUNT | repetition unit of learning |
| GPU_COUNT | number of GPUs |
| IMAGES_PER_GPU | number of GPUs assigned per |
| | image |
| STEPS_PER_STEPS | number of times of learning |
| | per EPOCH |
| VALIDATION_STEPS | number of times of validation |
| | learning |
| DETECTION_MIN_CONFIDENCE | detection threshold |
| LEARNING_RATE | learning accuracy |
| USE_MINI_MASK | minimum mask usage flag |
| RPN_ANCHOR_SCALES | learning and detection |
| | anchor scale size |

As another example, the prediction model 122 may apply a data learning algorithm and perform data learning. Specifically, a server for image learning may be constructed and a quality result report for the whole data set may be created.

For example, the quality result report for the whole dataset may be shown as in Table 6.

TABLE 6

| Train | | Detect | |
|---|---|---|---|
| 1. Rule | rulebio | 1. Rule | rulebio |
| Data | | Data | |
| file | | file (Baxh | |
| | | task | |
| | | name) | |
| 2. task | 20210114-135121 | 2. task | 20210115-090245 |
| start | | start | |
| time | | time | |
| 3. learning | bio | 3. test | bio |
| model | | image | |
| image | | type | |
| type | | | |
| 4. position | image/bio/it-220 | 4. position | image/bio/it-220 |
| of test | | of test | |
| data set | | data set | |
| 5. learning | train-1913 | 5. learning | test-747 |
| data set | validation-612 | data set | |
| size (number | | size (number | |
| of images) | | of images) | |
| 6. applied | EPOCH_COUNT = 50 | 6. applied | GPU_COUNT = 1 |
| hyper | GPU_COUNT = 2 | hyper | IMAGES_PER_GPU = 1 |
| parameter | IMAGES_PER_GPU = 1 | parameter | DETECTION_MIN_CONFIDENCE = [symbol] |
| | STEPS_PER_EPOCH = 1000 | | |
| | VALIDATION_STEPS = 100 | | |
| | DETECTION_MIN_CONFIDENCE = [symbol] | | |
| | LEARNING_RATE = [symbol] | | |
| | USE_MIN_MASK = False | | |
| | RP [symbol] _ANCHOR_SCALES ( [symbol] | | |
| | RP [symbol] _TRAIN_ANCHORS_PER_ IMAGE = | | |
| | [symbol] | | |
| 7. learning | bio-tr002-p001-e [symbol] | 7. used | bio-tr002-p001-e50 [symbol] |
| model | | model | |
| 8. learning | [symbol] | 8. test | number of detection |
| result | [symbol] | result | success images |
| | [symbol] | | number of detection |
| | [symbol] | | failure images |
| | [symbol] | | [symbol] |
| | [symbol] | | [symbol] |
| | [symbol] | | [symbol] |
| | [symbol] | | [symbol] |
| | [symbol] | | [symbol] |
| | [symbol] | | |
| | [symbol] | | |
| | [symbol] | | |
| 9. time | 25590 sec = 7.10 hour | 9. time | 519 sec |
| required | | required | |
| for | | for | |
| learning | | learning | |

[symbol]

indicates data missing or illegible when filed

Further, the prediction model 122 may use a convolutional neural network (CNN) algorithm as an algorithm for image data learning. Specifically, the CNN algorithm, together with the recurrent neural network (RNN), is attracting attention as one of the two major deep learning models, and may be basically based on the structure proposed by Jan Lekun in 1989.

For example, the AlexNet algorithm may be applied as the CNN algorithm, and may include a conv layer, a max-pooling layer, five dropout layers, three fully connected layers, and a nonlinearity function (ReLU, batch stochastic gradient descent).

Further, the GoogleNet algorithm may be applied as the CNN algorithm, and a conv layer to which one conv filter is applied may be simply deeply stacked, but individual layers may be thickly expanded by introducing various types of filters or pooling in one layer.

For example, the data construction scale may be shown as in Table 7.

TABLE 7

| staining method | data count | ratio |
|---|---|---|
| H&E | 1091 | 19.9% |
| Pap | 4410 | 80.1% |
| total | 5502 | 100% |

For example, the data learning algorithm performance may be calculated according to the artificial intelligence data utilization model development summary table, the validity validation environment, and the learning conditions.

For example, as the performance index of the prediction model 122, the accuracy was shown as 80% as in Table 8.

TABLE 8

| | | model | |
|---|---|---|---|
| | | performance | application |
| data name | AI model | index | service |
| cytology | R-CNN | Accuracy 80% | cytology |
| image patch | | | diagnosis AI |
| data set | | | system |

As an example, the validity validation environment and learning conditions may be shown as in Table 9.

TABLE 9
validity validation item

| | |
|---|---|
| item name | cancer diagnosis and detailed type |
| | diagnosis classification model |
| validation | Accuracy |
| method | |
| purpose | diagnosis classification of cancer |
| | and normal for collected samples |
| index | Accuracy 0.8 or more (80%) |
| measurement | Accuracy = (true positive/total |
| formula | case) *100 |

validity validation environment

| | |
|---|---|
| CPU | 40Core (2.4 GHz) |
| Memory | 503 GB |
| GPU | TITAN Xp 12 GP 8 count |
| Storage | HDD 11TB |
| OS | Linux |

validity validation model learning and
validation conditions

| | |
|---|---|
| used language | Python |
| framework | Pytorch |
| learning | [symbol] |
| algorithm | |
| learning | Loss: Binary cross Entropy |
| conditions | Optimizer: Adam |
| file format | learning data set: jpg |
| | evaluation data set: jpg |
| application to | AI model use image ratio (number) |
| model relative | more than one, normal: 100% (507 WSI, |
| to whole | 21, 527 sheets of image patches) |
| construction | more than one, cancer (524 WSI, |
| | 24, 542 sheets of image patches) |
| | *The whole constructed data applies |
| | to validity validation which is |
| | discussed with the person in charge |
| | of TTA if a change is required. |
| data | [model] |
| classification | training set ratio (number) |
| and ratio | 80% of all (823 WSI, 36,841 sheets of |
| information | image patches, normal 403 WSI, cancer |
| for each model | 420 WSI) |
| training | 10% of all (104 WSI 4, 606 sheets of |
| process | image patches, normal 52 WSI, cancer |
| | 52 WSI) |
| | 10% of all (104 WSI, 4, 605 sheets of |
| | image patches, normal 52 WSI, cancer |
| | 52 WSI) |

[symbol]

indicates data missing or illegible when filed

Performance is evaluated on the test data set among the established data sets, and the test data set and the basic data set should not overlap each other. Further, as described above, the prediction model 122 may be based on an annotation regarding whether the slide unit neoplasm is present.

As described above, the specimen cytology supporting device 100 may support collecting cytology specimens or glass slide specimens by the cell staining method, body fluid type, normal and cancer diagnosis class, and carcinoma, and normal/cancer or detailed dividing carcinoma according to the cell staining method.

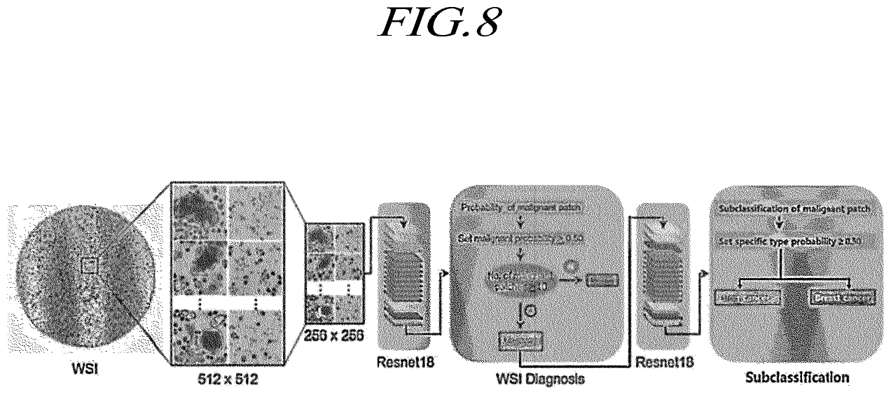

Referring to FIG. 8, after extracting an image patch having a size of 512×512 pixels from a slide image WSI and then resizing the extracted image patch to a size of 256×256 pixels, the specimen cytology supporting device 100 according to an embodiment may identify whether there is cancer according to a cell staining method using a first prediction model (WSI Diagnosis) for the image patches, and may identify the type of cancer using a second prediction model (subclassification) for the image patches.

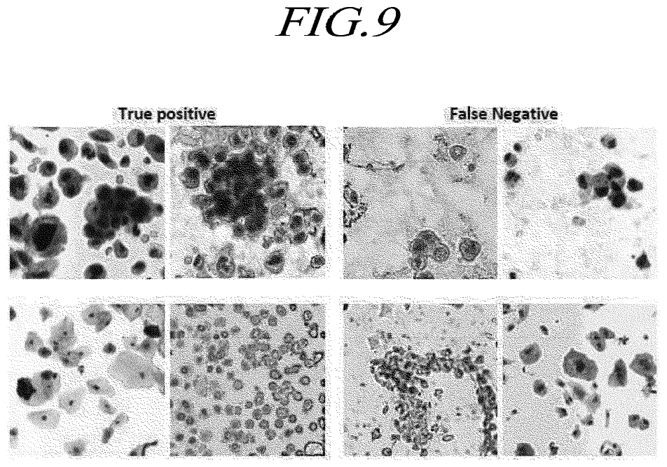

Accuracy may define a prediction score compared with a reference standard result of the prediction model 122 for each carcinoma as positive/negative based on a cutoff. A 2×2 table may be created based on the defined result. The created table may be shown as in Tables 10 and 11. FIG. 9 illustrates images corresponding to true positive and false negative.

TABLE 10

reference standard

| | | positive | negative | total |
|---|---|---|---|---|
| result | positive | true | false | true |
| | | positive | positive | positive + |
| | | | | false |
| | | | | positive |
| | negative | false | true | false |
| | | negative | negative | negative + |
| | | | | true |
| | | | | negative |
| | total | true | false | total |
| | | positive + | positive + | number of |
| | | false | true | specimens |
| | | negative | negative | |

TABLE 11

True class

| | | True | False |
|---|---|---|---|
| | | (Abnormal) | (Normal) |
| Predicted | True | 1394 | 437 |
| Class | (Abnormal) | | |
| | False | 238 | 1531 |
| | (Normal) | | |

Here, accuracy may mean the sum of true positive and true negative. Further, the accuracy of the total number of specimens may be between 0 and 100%, and the accuracy closer to 100% may mean more ideal diagnostic performance.

As another example, the obtained digital cytology slide image may be basically divided into training/validation/test data at a ratio of about 8:1:1 considering the quantity distribution for each class, and may be configured finally at a ratio of 8:1:3 considering the importance of the test data set.

### Embodiment

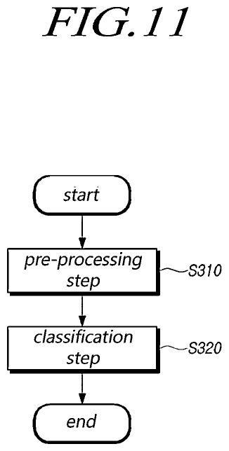

A learning algorithm was developed, as illustrated in FIG. 11A, by dividing data into training, validation, and evaluation in the form illustrated in Table 4 below.

The learning algorithm illustrated in FIG. 11A may classify a class of at least one of whether there is cancer and the type of cancer according to the cell staining method in any specimen cytology slide image using the prediction model that has undergone annotation-based learning using the specimen cytology slide image divided according to the cell staining method or the plurality of tile images.

Specifically, as illustrated in FIG. 11A, convolution, max polling, four residual blocks, and average pooling are performed on the input data, and output data is output in a sigmoid. As shown on the right side of FIG. 11A, the residual block may be implemented by an ReLU using two convolutions.

Table 12 illustrates the results of H&E staining and PAP staining among the cell staining methods by the learning algorithm illustrated in FIG. 11A. Table 13 illustrates the sensitivity, specificity, and accuracy of H&E staining and PAP staining according to the predicted results.

TABLE 12

Prediction

| | | H&E | PAP | Total |
|---|---|---|---|---|
| Ground | HE | 2427 | 0 | 2427 |
| Truth | PAP | 0 | 2429 | 2429 |

| | | | |
|---|---|---|---|
| Total | 2427 | 2429 | 4856 |

TABLE 13

| Label | H&E | PAP |
|---|---|---|
| Sensitivity | 1.0000 | 1.0000 |
| Specificity | 1.0000 | 1.0000 |
| Accuracy | 1.0000 | 1.0000 |

A learning model was developed by dividing data into training, validation, and evaluation in the form illustrated in Tables 14 and 15 below. Table 14 illustrates data obtained by H&E staining among the cell staining methods, and Table 15 illustrates data obtained by PAP staining among cell staining methods.

The learning algorithm illustrated in FIG. 11B may classify a class of at least one of whether there is cancer and the type of cancer according to the body fluid type in any specimen cytology slide image using the prediction model that has undergone annotation-based learning using the specimen cytology slide image divided according to the body fluid type or the plurality of tile images.

Specifically, as illustrated in FIG. 11B, after performing the stem algorithm on the input data, inception-ResNet-A and reduction-A are repeatedly performed three times, and then, adaptive average pooling is performed, and output data is output in a sigmoid. The stem algorism, inception-ResNet-A, and Reduction-A may perform filter concat after performing convolution and max polling continuously or repeatedly, as shown on the right side of FIG. 11B.

TABLE 14
Dataset (PAP)

| | Train | Valid | Test | Total |
|---|---|---|---|---|
| Respiratory | 19666 | 2458 | 2460 | 24584 |
| Pleura fluid | 33509 | 4189 | 4191 | 41889 |
| Ascites | 28095 | 3512 | 3517 | 35124 |
| FNA | 24177 | 3022 | 3027 | 30226 |
| Urine | 32389 | 4048 | 4050 | 40487 |
| Total | 137836 | 17229 | 17245 | 172310 |

TABLE 15

| | | | |
|---|---|---|---|
| Train | Valid | Test | Total |

Dataset (PAP)

| | | | | |
|---|---|---|---|---|
| Respiratory | 19666 | 2458 | 2460 | 24584 |
| Pleura fluid | 33509 | 4189 | 4191 | 41889 |
| Ascites | 28095 | 3512 | 3517 | 35124 |
| FNA | 24177 | 3022 | 3027 | 30226 |
| Urine | 32389 | 4048 | 4050 | 40487 |
| Total | 137836 | 17229 | 17245 | 172310 |

Dataset (HE)

| | | | | |
|---|---|---|---|---|
| Respiratory | 4804 | 601 | 601 | 6006 |
| Pleura fluid | 6376 | 798 | 798 | 7972 |
| Ascites | 8754 | 1094 | 1097 | 10945 |
| FNA | 7789 | 973 | 977 | 9739 |
| Urine | 0 | 0 | 0 | 0 |
| Total | 27723 | 3466 | 3473 | 34662 |

Tables 16 and 18 illustrate the results predicted according to the body fluid type by H&E staining and PAP staining among the cell staining methods by the learning algorithm illustrated in FIG. 11B. Tables 17 and 19 illustrate the sensitivity, specificity, and accuracy of H&E staining and PAP staining according to the results predicted according to the body fluid type.

TABLE 16

| | | Prediction | | | | | |
|---|---|---|---|---|---|---|---|
| | | 01_ | | | | | |
| | | RESP | 02_PF | 03_ASC | 04_FNA | 05_U | Total |
| Ground | 01_RESP | 2323 | 21 | 17 | 62 | 37 | 2460 |
| Truth | 02_PF | 12 | 4008 | 148 | 11 | 12 | 4191 |
| | 03_ASC | 16 | 198 | 3278 | 15 | 10 | 3517 |
| | 04_FNA | 41 | 6 | 21 | 2947 | 12 | 3027 |
| | 05_U | 30 | 17 | 9 | 23 | 3971 | 4050 |

| | | | | | | |
|---|---|---|---|---|---|---|
| Total | 2422 | 4250 | 3473 | 3058 | 4042 | 17245 |

TABLE 17

| Label | 01_RESP | 02_PF | 03_ASC | 04_FNA | 05_U |
|---|---|---|---|---|---|
| Sensitivity | 0.9443 | 0.9563 | 0.9320 | 0.9736 | 0.9805 |
| Specificity | 0.9933 | 0.9815 | 0.9858 | 0.9922 | 0.9946 |
| Accuracy | 0.9443 | 0.9563 | 0.9320 | 0.9736 | 0.9805 |

TABLE 18

| | | Prediction | | | | |
|---|---|---|---|---|---|---|
| | | 01_RESP | 02_PF | 03_ASC | 04_FNA | Total |
| Ground | 01_RESP | 599 | 2 | 0 | 0 | 601 |
| Truth | 02_PF | 8 | 767 | 22 | 1 | 798 |
| | 03_ASC | 1 | 13 | 1081 | 2 | 1097 |
| | 04_FNA | 7 | 5 | 23 | 942 | 977 |

| | | | | | |
|---|---|---|---|---|---|
| Total | 615 | 787 | 1126 | 945 | 3473 |

TABLE 19

| Label | 01_RESP | 02_PF | 03_ASC | 04_FNA |
|---|---|---|---|---|
| Sensitivity | 0.9967 | 0.9612 | 0.9854 | 0.9642 |
| Specificity | 0.9944 | 0.9925 | 0.9811 | 0.9988 |
| Accuracy | 0.9967 | 0.9612 | 0.9854 | 0.9642 |

According to the present embodiments, it is possible to provide a classification model for classifying four types of cancers including ovarian cancer, colon cancer, gastric cancer, and pancreatic cancer occurring in a specimen using an image obtained by digitally scanning a cytology glass slide of the specimen. By applying the classification model to specimen cytology specimen analysis, it is possible to provide a classification model that enables more accurate early diagnosis with a simple, chip, and non-invasive test.

FIG. 11 is a flowchart illustrating an example specimen cytology supporting method 200 according to another embodiment.

Referring to FIG. 11, a specimen cytology supporting method 200 according to another embodiment includes a pre-processing step S210 of extracting a plurality of tile images from a specimen cytology slide image divided according to a cell staining method and a classification step S220 of classifying a class of at least one of whether there is cancer or a type of cancer according to a cell staining method from the specimen cytology slide image divided according to any cell staining method using a prediction model in which annotation-based learning is performed on the specimen cytology slide image divided according to the cell staining method or the tile images.

As described above in connection with FIGS. 2 to 6, the specimen cytology slide image 10 divided according to the cell staining method may be obtained from the original slide image 12 obtained by spearing and capturing or scanning on the glass slide of the specimen using the Z-stacking or focus stacking technique.

Further, the specimen cytology slide image 10 divided according to the cell staining method may be obtained by synthesizing the images 14 focused at different phases from the original slide image 12 into one image 16 through secondary post-processing using Z-stacking or focus stacking technique.

In the pre-processing step S210, the plurality of tile images may be generated based on the sliding window algorithm.

As described above, the prediction model 122 that has undergone annotation-based learning may perform learning by adding one or more of partial annotation 32 indicating the cancer area in a line form, bounding box annotation 34 indicating the cancer area in a box form, and image-level label 36 indicating the whole image to the specimen cytology slide image divided according to the cell staining method or the plurality of tile images used for learning, as described above in connection with FIG. 7.

There are various cell staining methods used in cytology. Further, various cell staining methods have been developed and used to analyze specific diseases or cell structures. This may vary depending on the type of cell staining method and the purpose of the test.

For example, the cell staining method may be one of Giemsa staining, hematoxylin-eosin (H&E) staining, NissI staining, reticulin staining, Papanicolaou (PAP) staining, and Diff-quik staining.

A plurality of prediction models 122 that have undergone annotation-based learning may be present for each cell staining method of the specimen and for each type of cancer. In this case, the classification step S220 may classify a class of at least one of whether there is cancer or the type of cancer according to each cell staining method in any specimen cytology slide image using the prediction model 122 that has undergone the annotation-based learning for each cell staining method of the specimen and each type of cancer.

The prediction model 122 may perform a data gathering step and, as described above, the data pre-processing step, the model training step for performing annotation-based learning or weakly-supervised learning, and a model validation step for validating the trained model, analyze the specimen cytology slide image divided according to any cell staining method when the specimen cytology slide image divided according to any cell staining method is input, and classify a class of at least one of whether there is cancer and the type of cancer according to the cell staining method. The prediction model 122 may also identify the position of the arm using an image classification algorithm.

The prediction model 122 may divide whether there is cancer or the type of cancer according to the cell staining method into a respective class and define, and when receiving a request for the classification of the class for the specimen cytology slide image 10 divided according to any cell staining method, may classify the class as a result of annotation-based learning.

Further, the prediction model 122 may be generated using an ensemble learning method. One prediction model 122 may classify whether there is cancer and the type of cancer according to the above-described cell staining. There may be prediction models 122 of specimens trained for each cancer, and each prediction model 122 of specimens trained for each cancer may determine whether the cancer corresponds to the corresponding cancer, and the results of prediction by the prediction models may be compiled to classify whether there is cancer and the type of cancer according to the cell staining method.

For example, for the specimen cytology slide image 10 divided according to any cell staining method, the prediction model 122 trained with a specific cancer may classify the cancer as the corresponding cancer, and the prediction model 122 trained to classify detailed cancers may classify the carcinoma as one of the detailed cancers. The prediction model 122 trained with other cancers may classify the cancer as not corresponding to the corresponding cancer.

Further, the prediction model 122 may divide whether there is cancer or the type of cancer according to the type of body fluid into a respective class and define, and when receiving a request for the classification of the class for the specimen cytology slide image 10 divided according to any body fluid type, may classify the class as a result of annotation-based learning.

In other words, there are a plurality of prediction models that have undergone annotation-based learning according to the body fluid types of the specimen and the cancer types.

The classification step 220 may classify a class of at least one of whether there is cancer or the type of cancer according to the body fluid type in any specimen cytology slide image using the prediction model that has undergone the annotation-based learning for the body fluid type of the specimen and each type of cancer.

In this case, the specimen according to the type of body fluid may be at least one of a respiratory specimen, pleural fluid, ascites, urine, and fine needle aspiration cytology.

Whether there is cancer according to the type of body fluid may be divided into positive or negative.

If the specimen is a respiratory specimen, the type of cancer may be lung cancer, if the specimen is pleural fluid, the type of cancer may be lung cancer and breast cancer, if there are a plurality of specimens, the type of cancer may be ovarian cancer, colon cancer, gastric cancer, and pancreatic cancer, if the cancer is ovarian cancer, the cancer may be serous cancer, mucous cancer, endometrial cancer, or transparent cell cancer, if the urine, the type of cancer may be bladder specimen is cancer, and if the specimen is fine needle aspiration cytology, the type of cancer may be thyroid gland, salivary gland, lung cancer, pancreatic cancer, lymphoma, but is not limited thereto.

This prediction model 122 may classify only whether there is cancer according to the type of body fluid, or may classify the type of cancer as well as whether there is cancer.

For example, the prediction model 122 may classify only whether there is cancer according to the type of body fluid in the specimen, or may classify the type of cancer as well as whether there is cancer according to the type of body fluid.

For example, when the specimen is a respiratory specimen, the prediction model 122 may include a lung cancer classification model according to the cancer.

When the specimen is pleural fluid, the prediction model 122 may include a lung cancer and breast cancer classification model according to the cancer.

When there are a plurality of specimens, the prediction model 122 may include a classification model for ovarian cancer, colon cancer, gastric cancer, and pancreatic cancer according to the cancer. In particular, in the case of ovarian cancer, the prediction model 122 may include a serous cancer, mucous cancer, endometrial cancer, or transparent cell cancer classification model.

When the specimen is urine, the prediction model 122 may include a bladder cancer classification model according to cancer.

When the specimen is fine needle aspiration cytology, the prediction model 122 may include of a thyroid gland, salivary gland, lung cancer, pancreatic cancer, and lymphoma classification model according to the cancer.

The prediction model 122 may be a model for dividing each cancer. For example, when there are a plurality of specimens, the prediction model 122 may be a model for distinguishing whether it is ovarian cancer or not. The prediction model 122 may be a model for distinguishing whether it is colon cancer or not. The prediction model 122 may be a model for distinguishing whether it is gastric cancer or not. The prediction model 122 may be a model for distinguishing whether it is pancreatic cancer or not. In the case of ovarian cancer, another prediction model 122 may distinguish serous cancer, mucous cancer, endometrial cancer, or transparent cell cancer.

Even when the specimens are different, each prediction model 122 may be a model for distinguishing various types of cancers.

The description of the specimen cytology supporting device 100 according to an embodiment, made above in connection with FIGS. 1 to 6, may be likewise applied to the specimen cytology supporting method 200 according to another, described in connection with FIG. 11.

The corresponding specimen cytology supporting device and method according to the present embodiments may provide high accuracy and efficiency, thereby significantly helping to diagnose and treat the pathology test.

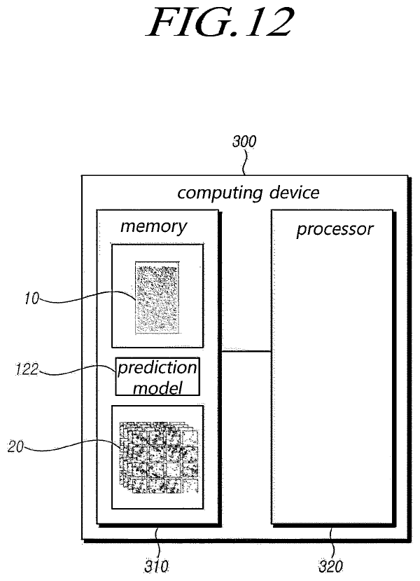

FIG. 12 is a block diagram illustrating a computing system 300 according to embodiments of the disclosure.

Referring to FIG. 12, a computing system 300 may include a memory 310 and a processor 320.

The memory 310 may store the specimen cytology slide image 10 divided according to the cell staining method and the plurality of tile images 20, but may also be separately stored in a separate large-capacity storage server or the like. The memory 310 may be a volatile memory (e.g., SRAM or DRAM) or nonvolatile memory (e.g., NAND Flash).

The processor 320 may extract a plurality of tile images from a specimen cytology slide image divided according to a cell staining method and classify a class of at least one of whether there is cancer or a type of cancer from the specimen cytology slide image divided according to any cell staining method using a prediction model in which annotation-based learning is performed on the specimen cytology slide image divided according to the cell staining method or the tile images.

The memory 310 stores the prediction model 122 that has undergone annotation-based learning. When receiving a request for classifying a class of at least one of whether there is cancer or the type of cancer in the specimen cytology slide image divided according to any cell staining method, the processor 320 executes the prediction model which has undergone annotation-based learning, stored in the memory 310, to classify a class of at least one of whether there is cancer or the type of cancer in the specimen cytology slide image divided according to the cell staining method and output the result.

The computing system according to embodiments of the disclosure may include a computer device 300 including a memory 310 and a processor 320, and a server 400 including a memory 410 and a processor 420. The computer device 300 and the server 400 may be wiredly or wirelessly connected through a network.

The memory 410 of the server 400 may store the above-described prediction model 122 that has undergone annotation-based learning.

When receiving a request (or query) for classifying a class of at least one of whether there is cancer or the type of cancer from the specimen cytology slide image divided according to any cell staining method, the processor 320 of the computer device 300 extracts a plurality of tile images from the specimen cytology slide image divided according to the cell staining method. The memory 310 of the computer device 300 may store the above-described specimen cytology slide image 10 divided according to the above-described cell staining method and the plurality of tile images 20.

The processor 320 of the computer device 300 may transmit the specimen cytology slide image 10 divided according to the cell staining method, the plurality of tile images 20, and the query stored in the memory 310 to the server 400.

The processor 420 of the server 400 may classify a class of at least one of whether there is cancer or the type of cancer in the received specimen cytology slide image divided according to the cell staining method or the specimen cytology slide image divided according to any cell staining method using the prediction model that has undergone annotation-based learning on the plurality of tile images, and transmit the result to the computer device 300.

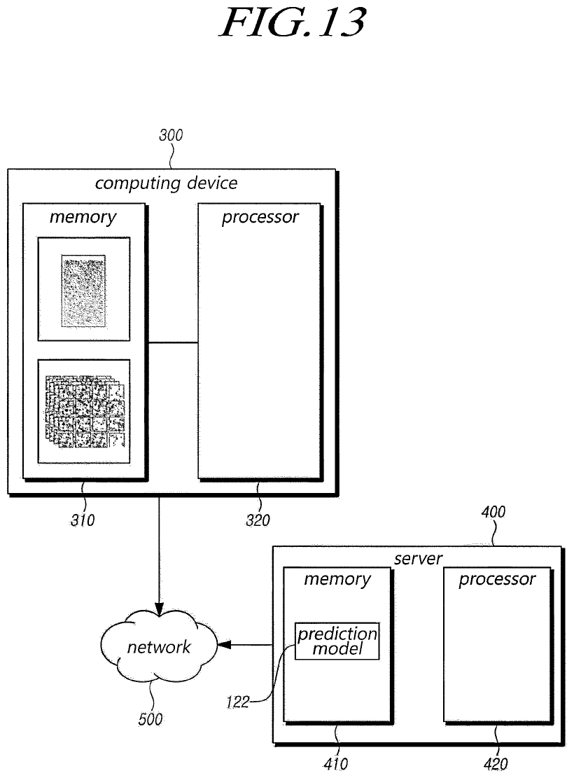

Various examples of the computer system described with reference to FIGS. 12 and 13 are described below.

The specimen cytology supporting device 200 may be configured as the computing system 300 illustrated in FIG. 12, or may be configured as a GPU server including storage for storing the scan file (WSI image), a GPU processor, and a general memory, but the disclosure is not limited thereto.

The above-described specimen cytology supporting device 100 may be implemented by a computing device including at least some of a processor, a memory, a user input device, and a presentation device. The memory is a medium that stores computer-readable software, applications, program modules, routines, instructions, and/or data, coded to perform specific tasks when executed by a processor. The processor may read and execute the computer-readable software, applications, program modules, routines, instructions, and/or data stored in the memory. The user input device may be a means for allowing the user to input a command to the processor to execute a specific task or to input data required for the execution of the specific task. The user input device may include a physical or virtual keyboard or keypad, key button, mouse, joystick, trackball, touch-sensitive input means, or a microphone. The presentation device may include, e.g., a display, a printer, a speaker, or a vibrator.

The computing device may include various devices, such as smartphones, tablets, laptops, desktops, servers, clients, and the like. The computing device may be a single stand-alone device and may include a plurality of computing devices operating in a distributed environment composed of a plurality of computing devices cooperating with each other through a communication network.

Meanwhile, the computing device may be a quantum computing device rather than a classical computing device. The quantum computing device performs operations in qubit units rather than bits. The qubit may have a state in which 0 and 1 overlap at the same time, and if there are M qubits, 2\hat{ }M states may be expressed at the same time.

The quantum computing device may use various types of quantum gates (e.g., Pauli/Rotation/Hadamard/CNOT/SWAP/Toffoli) that receive one or more qubits to perform a quantum operation and performs a designated operation and may configure a quantum circuit with a special function by combining the quantum gates.

The quantum computing device may use a quantum artificial neural network (e.g., QCNN or QGRNN) that may perform the functions performed by the conventional artificial neural network (e.g., CNN or RNN), using fewer parameters at higher speed.

Further, the above-described specimen cytology supporting device 100 may be executed by a computing device that includes a processor and a memory storing computer readable software, applications, program modules, routines, instructions, and/or data structures, coded to perform a specimen cytology supporting method utilizing a deep learning model when executed by the processor.

The present embodiments described above may be implemented through various means. For example, the present embodiments may be implemented by various means, e.g., hardware, firmware, software, or a combination thereof.

When implemented in hardware, the specimen cytology supporting method 200 using a deep learning model according to the present embodiments may be implemented by, e.g., one or more application specific integrated circuits (ASICS), digital signal processors (DSPs), digital signal processing devices (DSPDs), programmable logic devices (PLDs), field programmable gate arrays (FPGAs), processors, controllers, micro-controllers, or micro-processors.

For example, the specimen cytology supporting method 200 according to embodiments may be implemented by an artificial intelligence semiconductor device in which neurons and synapses of the deep neural network are implemented with semiconductor devices. In this case, the semiconductor devices may be currently available semiconductor devices, e.g., SRAM, DRAM, or NAND or may be next-generation semiconductor devices, such as RRAM, STT MRAM, or PRAM, or may be combinations thereof.

When the specimen cytology supporting method 200 according to embodiments is implemented using an artificial intelligence semiconductor device, the results (weights) of training the deep learning model with software may be transferred to synaptic mimic devices disposed in an array, or learning may be performed in the artificial intelligence semiconductor device.

When implemented in firmware or hardware, the specimen cytology supporting method 200 according to the present embodiments may be implemented in the form of a device, procedure, or function performing the above-described functions or operations. The software code may be stored in a memory unit and driven by a processor. The memory unit may be positioned inside or outside the processor to exchange data with the processor by various known means.

The above-described terms, such as “system,” “processor,” “controller,” “component,” “module,” “interface,” “model,” or “unit,” described above may generally refer to computer-related entity hardware, a combination of hardware and software, software, or software being executed. For example, the above-described components may be, but are not limited to, processes driven by a processor, processors, controllers, control processors, entities, execution threads, programs, and/or computers. For example, both an application being executed by a controller or a processor and the controller or the processor may be the components. One or more components may reside within a process and/or thread of execution, and the components may be positioned in one device (e.g., a system, a computing device, etc.) or distributed in two or more devices.

Meanwhile, another embodiment provides a computer program stored in a computer recording medium for performing the above-described specimen cytology supporting method 200. Further, another embodiment provides a computer-readable recording medium storing a program for realizing the above-described method for analyzing specimen cytology slide images divided according to the cell staining method.

The program recorded on the recording medium may be read, installed, and executed by a computer to execute the above-described steps.

As such, for the computer to read the program recorded on the recording medium and execute the implemented functions with the program, the above-described program may include code coded in a computer language, such as C, C++, JAVA, or machine language, which the processor (CPU) of the computer may read through a computer device interface.

Such code may include a function code related to a function defining the above-described functions or may include an execution procedure-related control code necessary for the processor of the computer to execute the above-described functions according to a predetermined procedure.

Further, the code may further include additional information necessary for the processor of the computer to execute the above-described functions or memory reference-related code as to the position (or address) in the internal or external memory of the computer the media should reference.

Further, when the processor of the computer needs to communicate with, e.g., another computer or a server at a remote site to execute the above-described functions, the code may further include communication-related code as to how the processor of the computer should communicate with the remote computer or server using the communication module of the computer and what information or media should be transmitted/received upon communication.

The above-described computer-readable recording medium may include, e.g., ROMS, RAMS, CD-ROMs, magnetic tapes, floppy disks, or optical data storage devices, or may also include carrier wave-type implementations (e.g., transmissions through the Internet).

Further, the computer-readable recording medium may be distributed to computer systems connected via a network, and computer-readable codes may be stored and executed in a distributed manner.

The functional programs for implementing the disclosure and code and code segments related thereto may easily be inferred or changed by programmers of the technical field to which the disclosure pertains, considering, e.g., the system environments of the computer reading and executing the program.

The specimen cytology supporting method 200 described in connection with FIG. 11 may be implemented in the form of recording media including computer-executable instructions, such as application or program modules. The computer-readable medium may be an available medium that is accessible by a computer. The computer-readable storage medium may include a volatile medium, a non-volatile medium, a separable medium, and/or an inseparable medium. The computer-readable medium may include a computer storage medium. The computer storage medium may include a volatile medium, a non-volatile medium, a separable medium, and/or an inseparable medium that is implemented in any method or scheme to store computer-readable commands, data architecture, program modules, or other data or information.

The above-described specimen cytology supporting method 200 may be executed by an application installed on a terminal, including a platform equipped in the terminal or a program included in the operating system of the terminal), or may be executed by an application (or program) installed by the user on a master terminal via an application providing server, such as a web server, associated with the service or method, an application, or an application store server. In such a sense, the above-described specimen cytology supporting method 200 may be implemented in an application or program installed as default on the terminal or installed directly by the user and may be recorded in a recording medium or storage medium readable by a terminal or computer.

Although embodiments of the disclosure have been described with reference to the accompanying drawings, it will be appreciated by one of ordinary skill in the art that the disclosure may be implemented in other various specific forms without changing the essence or technical spirit of the disclosure. Thus, it should be noted that the above-described embodiments are provided as examples and should not be interpreted as limiting. Each of the components may be separated into two or more units or modules to perform its function(s) or operation(s), and two or more of the components may be integrated into a single unit or module to perform their functions or operations.

It should be noted that the scope of the disclosure is defined by the appended claims rather than the described description of the embodiments and include all modifications or changes made to the claims or equivalents of the claims.

The above-described embodiments are merely examples, and it will be appreciated by one of ordinary skill in the art various changes may be made thereto without departing from the scope of the disclosure. Accordingly, the embodiments set forth herein are provided for illustrative purposes, but not to limit the scope of the disclosure, and should be appreciated that the scope of the disclosure is not limited by the embodiments. The scope of the disclosure should be construed by the following claims, and all technical spirits within equivalents thereof should be interpreted to belong to the scope of the disclosure.

What is claimed is:

1. A specimen cytology supporting device according to a cell staining method, comprising:
a pre-processor extracting a plurality of tile images from a specimen cytology slide image divided according to the cell staining method; and
a classifier classifying a class of at least one of whether there is cancer and a type of cancer according to the cell staining method in any specimen cytology slide image using a prediction model that has undergone annotation-based using the specimen cytology slide image divided according to the cell staining method or the plurality of tile images.

2. The specimen cytology supporting device of claim 1, wherein the cell staining method may be one of Giemsa staining, hematoxylin-eosin (H&E) staining, NissI staining, reticulin staining, Papanicolaou (PAP) staining, and Diff-quik staining.

3. The specimen cytology supporting device of claim 2, wherein there are a plurality of prediction models that have undergone annotation-based learning for each cell staining method of the specimen and for each type of cancer, and
wherein the classifier classifies a class of at least one of whether there is cancer or the type of cancer according to each cell staining method in any specimen cytology slide image using each prediction model that has undergone the annotation-based learning for each cell staining method of the specimen and for each type of cancer.

4. The specimen cytology supporting device of claim 1, wherein the specimen cytology slide image is obtained by applying Z-stacking or focus stacking to an original slide image obtained by spearing and capturing or scanning on a glass slide of the specimen.

5. The specimen cytology supporting device of claim 4, wherein the specimen cytology slide image is obtained by synthesizing images focused at different phases from the original slide image into one image through secondary post-processing, using Z-stacking or focus stacking.

6. The specimen cytology supporting device of claim 1, wherein the pre-processor generates the plurality of tile images based on a sliding window algorithm.

7. The specimen cytology supporting device of claim 1, wherein the prediction model that has undergone the annotation-based learning undergoes learning by adding one or more of a partial annotation indicating a cancer area in a line form, a bounding box annotation indicating the cancer area in a box form, and an image-level label indicating a whole image to the specimen cytology slide image divided according to the cell staining method or the plurality of tile images used for learning.

8. A specimen cytology supporting method according to a cell staining method, comprising:
a pre-processing step extracting a plurality of tile images from a specimen cytology slide image divided according to the cell staining method; and
a classification step classifying a class of at least one of whether there is cancer and a type of cancer according to the cell staining method in any specimen cytology slide image using a prediction model that has undergone annotation-based learning using the specimen cytology slide image divided according to the cell staining method or the plurality of tile images.

9. The specimen cytology supporting method of claim 8, wherein the cell staining method may be one of Giemsa staining, hematoxylin-eosin (H&E) staining, NissI staining, reticulin staining, Papanicolaou (PAP) staining, and Diff-quik staining.

10. The specimen cytology supporting method of claim 9, wherein there are a plurality of prediction models that have undergone annotation-based learning for each cell staining method of the specimen and for each type of cancer, and
wherein the classification step classifies a class of at least one of whether there is cancer or the type of cancer according to each cell staining method in any specimen cytology slide image using each prediction model that has undergone the annotation-based learning for each cell staining method of the specimen and for each type of cancer.

11. The specimen cytology supporting method of claim 8, wherein the specimen cytology slide image is obtained by applying Z-stacking or focus stacking to an original slide image obtained by spearing and capturing or scanning on a glass slide of the specimen.

12. The specimen cytology supporting method of claim 11, wherein the specimen cytology slide image is obtained by synthesizing images focused at different phases from the original slide image into one image through secondary post-processing, using Z-stacking or focus stacking.

13. The specimen cytology supporting method of claim 8, wherein the pre-processing step generates the plurality of tile images based on a sliding window algorithm.

14. The specimen cytology supporting method of claim 8, wherein the prediction model that has undergone the annotation-based learning undergoes learning by adding one or more of a partial annotation indicating a cancer area in a line form, a bounding box annotation indicating the cancer area in a box form, and an image-level label indicating a whole image to the specimen cytology slide image divided according to the cell staining method or the plurality of tile images used for learning.

15. A computer device, comprising:
a memory storing a specimen cytology slide image divided according to a cell staining method, a plurality of tile images extracted from the specimen cytology slide image, and a prediction model, the prediction model being a prediction model that has undergone annotation-based learning to classify a class of at least one of whether there is cancer or a type of cancer according to the cell staining method in any specimen cytology slide image using the specimen cytology slide image divided according to the cell staining method or the plurality of tile images; and
a processor, when receiving a request for classifying a class of at least one of whether there is cancer and the type of cancer in any specimen cytology slide image, extracting the plurality of tile images from the specimen cytology slide image, and executing the prediction model that has undergone the annotation-based learning stored in the memory to classify a class of at least one of whether there is cancer or the type of cancer according to the cell staining method in the specimen cytology slide image.
